(19) United States
(12) Patent Application Publication
(10) Pub. No.: US 2019/0247520 A1
(43) Pub. Date: Aug. 15, 2019

(54) NON-INVASIVE DETECTION OF DISEASE PROGRESSION AND THERAPEUTIC EFFICACY FOR MUSCULAR DYSTROPHIES AND DISEASES

(71) Applicants: Carl A. Morris, Woburn, MA (US); Kathryn Rae Wagner, Baltimore, MD (US)

(72) Inventors: Carl A. Morris, Woburn, MA (US); Kathryn Rae Wagner, Baltimore, MD (US)

(21) Appl. No.: 16/256,763

(22) Filed: Jan. 24, 2019

Related U.S. Application Data
(60) Provisional application No. 62/621,491, filed on Jan. 24, 2018.

(51) Int. Cl. A61K 49/00; A61K 49/06; A61K 51/02

(52) U.S. Cl. CPC A61K 49/0004; A61K 51/02; A61K 49/006; A61K 49/06

(57) ABSTRACT

Provided herein are non-invasive in vivo imaging methods to evaluate efficacy of a therapeutic intervention for neuromuscular diseases by determining the level and/or localization of a detectable marker that is influenced by another protein which is a target of the therapeutic intervention. Non-invasive imaging methods disclosed herein can involve in vivo molecular imaging such as PET or SPECT, and/or morphological imaging such as MRI or CT.

### RELATED APPLICATIONS

This application claims the benefit under 35 U.S.C. § 119(e) from U.S. provisional application No. 62/621491, filed Jan. 24, 2018, the entire contents of which are incorporated by reference herein.

### BACKGROUND

A considerable concern in clinical studies for disease correcting therapeutic interventions for neuromuscular diseases is the necessity of tissue biopsies (e.g., muscle biopsies in muscular dystrophy) to determine whether the therapeutic intervention is efficacious. This limitation of needing invasive biopsies results in poor subject recruitment for clinical trials and low sample numbers. For such neuromuscular diseases, monitoring of disease progression can also prove to be difficult.

### SUMMARY

The present application provides a non-invasive approach to make molecular level observations that provide a significant benefit not only to evaluate the efficacy of potential therapeutic interventions, but also to monitor neuromuscular disease progression. This approach to simply, effectively and non-invasively measure change at a molecular level, as provided herein, either alone or in combination with a morphological imaging technique, is also a means to identify and develop molecular markers that can predict eventual clinical and functional change in a subject. Biomarkers are useful in both current clinical trials and also as diagnostics in post-approval stages of drug discovery to continue monitoring effectiveness of a drug. The present application provides methods useful for gathering information on the status of a therapeutic target (e.g., level or functionality), by non-invasively measuring the level and/or localization of a detectable marker (e.g., a biomarker) that is affected by change in the therapeutic target.

In some aspects, provided herein is a method of evaluating efficacy of a therapeutic intervention in a subject suffering from a neuromuscular disease. In some embodiments, the method comprises determining level and/or localization of a detectable marker, the level and/or localization of which is modulated by a therapeutic target, by performing non-invasive in vivo molecular imaging on the subject after the subject undergoes the therapeutic intervention. In some embodiments, the detectable marker is a protein that is different from the therapeutic target.

Herein, a “detectable marker” (e.g., a biomarker) is a molecule (e.g., a protein) for which an imaging strategy is designed to detect. A “therapeutic target” is a molecule (e.g., a protein) for which a therapeutic strategy is designed to affect as a means to alter a neuromuscular disease or dystrophy.

In some embodiments, the method further comprises performing morphological imaging on the subject. In contrast to in vivo molecular imaging, morphological imaging provides information of a subjects tissue (e.g., a particular muscle) rather than of a particular molecule (e.g., a biomarker). Morphological imaging in conjunction with in vivo molecular imaging can be useful for providing a reference for the localization of a detectable marker. In some embodiments, morphological imaging is performed by the same imager that is used to perform in vivo molecular imaging.

Of course, in vivo molecular imaging may be performed on a subject after the subject undergoes a therapeutic intervention. However, in some embodiments, the method further comprises performance of in vivo molecular imaging on the subject before the subject undergoes the therapeutic intervention (e.g., before and after a therapeutic intervention).

In some embodiments, the level and/or localization of more than one detectable marker (e.g., a plurality of biomarkers) is determined.

In some embodiments, the detectable marker is a protein. In some embodiments, the detectable marker is a modified protein. In some embodiments, the modification of the modified detectable marker is glycosylation. In some embodiments, the detectable marker is a modified form of the therapeutic target (e.g., a glycosylated form of the therapeutic target).

In some embodiments, the therapeutic target is a cytosolic protein, a membrane-bound protein, a glycoprotein or a matrix-anchoring protein.

In some embodiments, the level and/or localization of the detectable marker is modulated by expression or activity of the therapeutic target. In some embodiments, a detectable marker binds to some form of a therapeutic target (e.g., to a functional form of the therapeutic target).

In some embodiments, the neuromuscular disease is one of the following: muscular dystrophy, spinal muscular atrophy, inflammatory myopathy, disease of peripheral nerve, disease of neuromuscular junction, metabolic disease of muscle, central core disease, hyperthyroid myopathy, myotonia congenita, myotubular myopathy, nemaline myopathy, paramyotonia congenita, periodic paralysis-hypokalemic-hyperkalemic myopathy, motor neuron disease, frailty syndrome or a condition associated with loss of muscle mass or function. In some embodiments, the muscular dystrophy is Becker muscular dystrophy, congenital muscular dystrophy, Duchenne muscular dystrophy (DMD), distal muscular dystrophy, Emery-Dreifuss muscular dystrophy, faciocarpulohumeral muscular dystrophy, limb-girdle muscular dystrophy (LGMD), myotonic muscular dystrophy, or oculopharyngeal muscular dystrophy.

In some embodiments, the frailty syndrome is sarcopenia. In some embodiments, the condition associated with loss of muscle mass or function is cachexia, chronic obstructive pulmonary disease (COPD), chronic kidney disease (CKD), heart failure, aging or acute muscle atrophy caused by immobilization due to injury or surgical procedure. In some embodiments, the injury is hip fracture or the surgical procedure is knee replacement.

In some embodiments, the therapeutic intervention is a pharmacologic, biologic or rehabilitative therapeutic intervention. In some embodiments, the therapeutic intervention is one that results in the increased expression or increased activity of a functional form of the therapeutic target. In some embodiments, the pharmacologic therapeutic intervention comprises administering to the subject a small molecule. In some embodiments, the biologic intervention comprises administering to the subject a biological molecule (e.g., a gene or protein, or virus particles harboring a gene). In some embodiments, the therapeutic intervention directly affects the level and/or localization of the detectable marker (e.g., a biomarker).

In some embodiments, the muscular dystrophy is Becker muscular dystrophy or Duchenne muscular dystrophy (DMD). In some embodiments, the muscular dystrophy is Duchenne muscular dystrophy (DMD). In some embodiments, the therapeutic target is dystrophin or utrophin. In some embodiments, the therapeutic target is dystrophin. In some embodiments, the therapeutic intervention is microdystrophin delivery, exon skipping antisense oligonucleotides for a gene encoding dystrophin, termination codon read through strategy for dystrophin, or gene editing for a gene encoding dystrophin. In some embodiments, the therapeutic target is utrophin. In some embodiments, the therapeutic intervention is micro-utrophin delivery, utrophin upregulation, membrane stabilization or α7 integrin modulation. In some embodiments, membrane stabilization is performed by upregulation of expression of galectin-1, biglycan, α7 integrin and/or laminin.

In some embodiments, the muscular dystrophy is Becker muscular dystrophy or DMD, and the detectable marker is a member of the dystrophin-associated glycoprotein complex (DGC). In some embodiments, the detectable marker is an extracellular matrix protein that binds to any member of the DGC. In some embodiments, the detectable marker is selected from the group consisting of: α-sarcoglycan, β-sarcoglycan, γ-sarcoglycan, δ-sarcoglycan, ε-sarcoglycan, ζ-sarcoglycan, α-dystroglycan, β-dystroglycan, laminin α2, α-syntrophin, β1-syntrophin, β-syntrophin, γ1-syntrophin, γ2-syntrophin, α-dystrobrevin, β-dystrobrevin, sarcospan, calveolin-3, and neuronal nitric oxide synthase (nNOS). In some embodiments, the muscular dystrophy is Becker muscular dystrophy or DMD, the therapeutic target is dystrophin or utrophin, and the detectable marker is selected from the group consisting of: α-sarcoglycan, γ-sarcoglycan, α-dystroglycan, β-dystroglycan and laminin α2.

In some embodiments, the limb-girdle muscular dystrophy (LGMD) is a LGMD1 or a LGMD2. In some embodiments, the LGMD1 is LGMD1A, LGMD1B, LGMD1C, LGMD1D, LGMD1E, LGMD1F, LGMD1G or LGMD1H; and the LGMD2 is LGMD2A, LGMD2B, LGMD2C, LGMD2D, LGMD2E, LGMD2F, LGMD2G, LGMD2H, LGMD2I, LGMD2J, LGMD2K, LGMD2L, LGMD2M, LGMD2N, LGMD2Q or LGMD2Q.

In some embodiments, the LGMD2 is LGMD2C, and the therapeutic target is γ-sarcoglycan. In some embodiments, the LGMD2 is LGMD2D, and the therapeutic target is α-sarcoglycan. In some embodiments, the LGMD2 is LGMD2E, and the therapeutic target is β-sarcoglycan. In some embodiments, the LGMD2 is LGMD2F, and the therapeutic target is δ-sarcoglycan. In some embodiments, the LGMD2 is LGMD2I, LGMD2K, LGMD2M, LGMD2N or LGMD2O; and the therapeutic target is a Fukutin kinase related protein (FKRP), POMT1, Fukutin, POMT2 or POMGnT 1. In some embodiments, the LGMD2 is LGMD2I, LGMD2K, LGMD2M, LGMD2N or LGMD2O, the therapeutic target is a Fukutin kinase related protein (FKRP), POMT1, Fukutin, POMT2 or POMGnT1, and the therapeutic intervention is delivery of Fukutin related protein (FKRP), POMT1, Fukutin, POMT2 or POMGnT1. In some embodiments, the detectable marker is glycosylated α-dystroglycan.

In some embodiments, the muscular dystrophy is myotonic muscular dystrophy. In some embodiments, the therapeutic target is muscleblind-like 1 (MBNL1) and the detectable marker is a membrane protein. In some embodiments, the membrane protein is chloride channel CIC-1 or insulin receptor. In some embodiments, the therapeutic intervention is upregulation of MBNL1 expression or MBNL1 release from RNA. In some embodiments, the myotonic muscular dystrophy is myotonic dystrophy type 1(DM1).

In some embodiments, the motor neuron disease is amyotrophic lateral sclerosis (ALS). In some embodiments, the therapeutic target is Dox7. In some embodiments, the detectable marker is a neuromuscular junction protein.

In some embodiments, the neuromuscular dystrophy is spinal muscular atrophy, the therapeutic target is survival of motor neuron (SMN), and the detectable marker is a neuromuscular junction protein.

In some embodiments, the neuromuscular junction protein is muscle specific kinase (MuSK), a lipoprotein receptor-related protein (LRP), or neural agrin.

In some embodiments, the non-invasive in vivo molecular imaging is selected from the group consisting of: positron emission tomography (PET), single-photon emission computer tomography (SPECT), magnetic resonance imaging (MRI), targeted contrast enhanced ultrasound (targeted CEUS) imaging and optical coherence tomography (OCT). In some embodiments, the morphological imaging is magnetic resonance imaging (MRI) or computerized tomography (CT). In some embodiments, the in vivo molecular imaging and morphological imaging is performed on the subject's upper or lower limb, upper thigh, or whole body. In some embodiments, the detectable marker is detected using an imaging agent with suitable affinity and specificity to the detectable marker. In some embodiments, the imaging agent is selected from the group consisting of: an antibody, a protein, a peptide, a small molecule, an antibody-derived construct, an adnectin, an aptamer, a nanobody and a protein domain. In some embodiments, the imaging agent is conjugated to a tracer. A tracer is a molecule capable of providing detectable signal. In some embodiments, the imaging agent is conjugated to a PET tracer, a SPECT tracer, a paramagnetic contrast agent, an ultrasound contrast agent or an OCT contrast agent. In some embodiments, the PET tracer is selected from the group consisting of: carbon-11, nitrogen-13, oxygen-15, fluorine-18, indium-11, gallium-68, copper-64, zirconium-89, iodine-125 and rubidium-82. In some embodiments, the SPECT tracer is selected from the group consisting of: iodine-123, technetium-99m, xenon-133, thallium-201 and fluorine-18. In some embodiments, the paramagnetic contrast agent is selected from the group consisting of: gadoterate, gadodiamide, gadobenate, gadopentetate, gadoteridol, gadofosveset, gadoversetamide, gadoxetate, gadobutrol and ferumoxsil.

In some aspects, provided herein is a method of monitoring a neuromuscular disease. The method can comprise determining level and/or localization of a detectable marker by performing in vivo molecular imaging on a subject. In some embodiments, the level and/or localization of the detectable marker is modulated by a therapeutic target. In some embodiments, the detectable marker is a modified form of the therapeutic target (e.g., a glycosylated form of the therapeutic target).

In some embodiments, the method of monitoring a neuromuscular disease further comprises performing morphological imaging on the subject.

In some embodiments, the method of monitoring a neuromuscular disease further comprises determining the level and/or localization of the detectable marker in the subject at two or more time points (e.g., separated by hours, days, months or years).

In some embodiments, the method further comprises determining the level and/or localization of the detectable marker in additional subjects with the same or different genetic abnormalities. Such measurements can be useful to establish prognosis or severity of a neuromuscular disease based on the genetic abnormality of a subject.

Any one of the methods of monitoring a neuromuscular disease as provided herein may further comprise determining the level and/or localization of the detectable marker after a therapeutic candidate has been administered to the subject, to determine the efficacy of the therapeutic candidate on clinical improvement.

In some embodiments, a therapeutic intervention is provided (e.g., administered) to a subject and the neuromuscular disease is then monitored in the subject using any one of the methods described herein to monitor the subject's neuromuscular disease.

In some embodiments of any one of the methods of monitoring a neuromuscular disease described herein comprises administering to the subject an imaging agent. In some embodiments, an imaging agent is an antibody, a protein, a peptide, a small molecule, an antibody-derived construct (e.g., scfv, Fab or Fab2′), an adnectin, an aptamer, a nanobody or a protein domain that has suitable affinity and specificity to a detectable marker to be detected. In some embodiments, an imaging agent is administered before determining the level and/or localization of a detectable marker comprising performing in vivo molecular imaging on a subject. In some embodiments, an imaging agent is administered simultaneously with determining the level and/or localization of a detectable marker comprising performing in vivo molecular imaging on a subject.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the embodiments illustrated in the drawings in no way limit the scope of the disclosure.

FIG. 1A and FIG. 1B depict a platform technology that provides a method to determine level and/or localization of specific proteins using a non-invasive detection method, which can be used to evaluate efficacy of a therapeutic intervention. FIG. 1A depicts a configuration in which a therapeutic intervention results in change of a therapeutic target (cylinder), which then results in change of level and/or localization of a detectable marker (rectangle) that can be detected using a non-invasive imaging technique to detect an imaging agent (star). FIG. 1B depicts a configuration in which a therapeutic intervention affects the level and/or localization of a detectable marker directly.

FIG. 2, which is adapted from Lisi and Cohn (Biochim Biophys Acta. 2007 February; 1772(2):159-72) and modified, depicts proteins involved in congenital muscular dystrophies, their localization and interactions: Laminin α-2, integrin α-7, collagen VI, α-dystroglycan, glycosyltransferases POMT1, POMT2, POGnT1, fukutin, FKRP and LARGE, and selenoprotein-N. ER: endoplasmic reticulum.

### DETAILED DESCRIPTION

One of the biggest difficulties in developing therapeutic interventions for neuromuscular diseases, including muscular dystrophies, is that there do not exist methods that are not tedious or invasive to evaluate the efficacy of a therapeutic intervention. For the same reason, determination of how far a neuromuscular disease has progressed is also difficult. For example, it is known that Duchenne muscular dystrophy (DMD) is caused by lack of functional dystrophin in muscles, which causes destabilization of the dystrophin-associated glycoprotein complex (DGC). However, there do not exist non-invasive methods to assess how much the DGC has destabilized.

In some aspects, this application provides a non-invasive method of inferencing the status of a cause of a neuromuscular disease or dystrophy (e.g., destabilization of DGC in DMD) that thus provides a more accurate measure of disease prognosis or the efficacy of a therapeutic intervention or candidate for therapeutic intervention (e.g., microdystrophin delivery as a treatment for DMD). In some embodiments, the disclosed methods, which involve the non-invasive measurement of a detectable marker, the level and/or localization of which is affected by a therapeutic target for a neuromuscular disease, provides a means to assess clinical or functional improvement. Such an assessment is useful for the development of new therapeutic strategies, as well as to determine severity of disease or monitor disease progression. It is also useful to determine whether a subject should be treated or determine the treatment strategy.

To that end, provided herein are methods to determine severity of disease, monitor disease progression, and evaluate efficacy of a therapeutic intervention or candidate for a therapeutic intervention for neuromuscular diseases and dystrophies. Methods disclosed herein involve imaging of a particular part of a subject's body or the entire body.

### Evaluating Efficacy of a Therapeutic Intervention

In some aspects, this disclosure provides a method of evaluating efficacy of a therapeutic intervention in a subject suffering from neuromuscular disease. In some embodiments, the method comprises determining a level and/or localization of a detectable marker (e.g., a biomarker) using in vivo molecular imaging on the subject after the subject undergoes a therapeutic intervention, wherein the level and/or localization of the detectable marker is modulated by a therapeutic target of the therapeutic intervention.

Herein, in vivo molecular imaging refers to in vivo imaging techniques with which a particular molecule can be detected in vivo. One advantage of such imaging techniques is that they are non-invasive, which renders both recruiting of subjects and obtaining approval of a protocol for evaluation of a therapeutic efficacy (e.g., by the US Food and Drug administration) easier to get achieve. Herein, “in vivo molecular imaging” is also referred to as “non-invasive in vivo imaging”, “molecular imaging”, “non-invasive molecular imaging”, “non-invasive in vivo molecular imaging.”

A “detectable marker” is a molecule for which an imaging strategy is designed to detect. A detectable marker is a molecule (e.g., a protein) that is detectable or that can be detected using imaging techniques (e.g., a biomarker). In some embodiments, the detectable marker is an extracellular protein, a membrane-associated extracellular protein, or a membrane-associated cytosolic protein. In some embodiments, the detectable marker is an extracellular matrix protein, or an extracellular matrix-anchoring protein. In some embodiments, a detectable marker is a neuromuscular junction protein, or myotendinous junction protein (e.g., a laminin or an integrin). In some embodiments, the detectable marker is a cytosolic protein. In some embodiments, the detectable marker is a modified protein (e.g., a glycosylated protein, a prenylated protein, a myristoylated protein, or a palmitoylated protein). In some embodiments, a modification is glycosylation. In some embodiments, the detectable marker is a modified version of the therapeutic target, e.g., the detectable marker may be a glycosylated version of the therapeutic target.

A “therapeutic target” is a molecule (e.g., a protein) for which a therapeutic strategy is designed to affect as a means to alter a neuromuscular disease or dystrophy. In some embodiments, the therapeutic target is a cytosolic protein, a membrane-bound protein, or a matrix-anchoring protein. In some embodiments, the therapeutic target is a modified protein (e.g., a glycosylated protein, a prenylated protein, a myristoylated protein, or a palmitoylated protein).

In some embodiments, level and/or localization of a detectable marker is modulated by the level of a therapeutic target. In some embodiments, the stability of a therapeutic target modulates or influences level and/or localization of a detectable marker. In some embodiments, modification (e.g., a post-translational modification) of a therapeutic target modulates level and/or expression of a detectable marker. In some embodiments, only the level of a detectable marker is modulated by a therapeutic target. In some embodiments, only localization of a detectable marker is modulated by a therapeutic target. In some embodiments, both level and localization of a detectable marker is modulated by a therapeutic target. In some embodiments, level and/or localization of a detectable marker is modulated directly by a therapeutic intervention.

In some embodiments, a detectable marker and therapeutic target are involved in the same disease pathway and are causal agents in disease. In some embodiments, a detectable marker is not involved in disease pathology, but serves as a biomarker of functional therapeutic target, where the therapeutic target is involved in disease pathology. In some embodiments, a therapeutic target is a protein, the loss of which is associated to disease, and whose expression can be restored by a therapeutic intervention. Table 1 provides examples of detectable markers and therapeutic targets for some neuromuscular diseases.

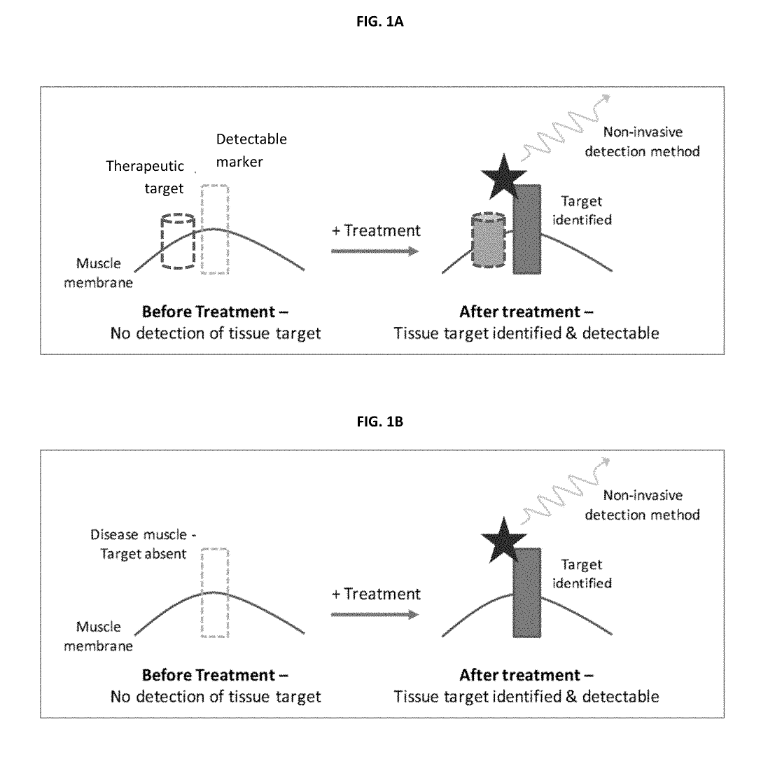

In some embodiments, a detectable marker is different from a therapeutic target. FIG. 1A illustrates such an embodiment. In some embodiments, as depicted in FIG. 1B, a detectable marker is directly affected by a therapeutic intervention. In some embodiments, a detectable marker is the same as a therapeutic target (for example, the detectable marker is the therapeutic target as well). For example, efficacy of a microdystrophin therapy can be evaluated by determining the level and/or localization of dystrophin itself in muscle. In some embodiments, a detectable marker binds directly with a therapeutic target. In some embodiments, a detectable marker and a therapeutic target do not bind directly, but are part of the same protein complex (e.g., DGC in muscular dystrophies such as DMD). In some embodiments, a detectable marker is not part of the protein complex to which the therapeutic target belongs, but binds to a member of the complex.

TABLE 1
Non-limiting examples of neuromuscular diseases, therapeutic interventions,
therapeutic targets, detectable markers, and organ specificity.

| | Therapeutic | Therapeutic | Detectable | |
|---|---|---|---|---|
| Indication | Intervention | Target | Marker | Organ Specificity |
| Duchenne MD | Microdystrophin | Dystrophin | Dystroglycan | Upper Thigh, |
| (DMD) | delivery (e.g., gene | | complex proteins | Whole Body |
| | therapy, mRNA, non- | | (e.g., alpha | |
| | viral) | | Sarcoglycan); | |
| | | | extracellular | |
| | | | matrix proteins | |
| | | | (e.g., laminin) | |
| | Exon skipping - | Dystrophin | Dystroglycan | Upper Thigh, |
| | antisense | | complex proteins | Whole Body |
| | oligonucleotides | | (e.g., alpha | |
| | | | Sarcoglycan); | |
| | | | extracellular | |
| | | | matrix proteins | |
| | | | (e.g., laminin) | |
| | Stop codon read- | Dystrophin | Dystroglycan | Upper Thigh, |
| | through | | complex proteins | Whole Body |
| | | | (e.g., alpha | |
| | | | Sarcoglycan); | |
| | | | extracellular | |
| | | | matrix proteins | |
| | | | (e.g., laminin) | |
| | Micro-utrophin | Utrophin | Dystroglycan | Upper Thigh, |
| | delivery (e.g., gene | | complex proteins | Whole Body |
| | therapy, mRNA, non- | | (e.g., alpha | |
| | viral) | | Sarcoglycan); | |
| | | | extracellular | |
| | | | matrix proteins | |
| | | | (e.g., laminin); | |
| | | | neuromuscular | |
| | | | junction proteins | |
| | | | e.g., MuSK, | |
| | | | LRP, neural | |
| | | | Agrin; | |
| | | | myotendinous | |
| | | | junction proteins | |
| | Utrophin | Utrophin | Dystroglycan | Upper Thigh, |
| | upregulation (e.g., | | complex proteins | Whole Body |
| | small molecules) | | (e.g., alpha | |
| | | | Sarcoglycan); | |
| | | | extracellular | |
| | | | matrix proteins | |
| | | | (e.g., laminin); | |
| | | | neuromuscular | |
| | | | junction proteins | |
| | | | e.g., MuSK, | |
| | | | LRP, neural | |
| | | | Agrin; | |
| | | | myotendinous | |
| | | | junction proteins | |
| | Membrane | Utrophin | Dystroglycan | Upper Thigh, |
| | stabilization (e.g., | | complex proteins | Whole Body |
| | galectin-1, biglycan, | | (e.g., alpha | |
| | alpha-7 integrin | | Sarcoglycan); | |
| | and/or laminin | | extracellular | |
| | upregulation) | | matrix proteins | |
| | Gene editing (e.g., | Dystrophin | Dystroglycan | Upper Thigh, |
| | CRISPR) | | complex proteins | Whole Body |
| | | | (e.g., alpha | |
| | | | Sarcoglycan) | |
| | alpha-7 integrin | Utrophin | Dystroglycan | Upper Thigh, |
| | modulation via small | | complex proteins | Whole Body |
| | molecule/other | | (e.g., alpha | |
| | | | Sarcoglycan); | |
| | | | extracellular | |
| | | | matrix proteins | |
| Becker MD | Same as DMD | Same as DMD | Same as DMD | Same as DMD |
| Limb-Girdle MD | Gene therapy/ | gamma- | Dystroglycan | Upper Thigh, |
| 2C | mRNA/Gene | sarcoglycan | complex proteins | Whole Body |
| | Editing | | (e.g., alpha | |
| | | | Sarcoglycan); | |
| | | | extracellular | |
| | | | matrix proteins | |
| Limb-Girdle MD | Gene therapy/ | alpha- | Dystroglycan | Upper Thigh, |
| 2D | mRNA/Gene | sarcoglycan | complex proteins | Whole Body |
| | Editing | | (e.g., alpha | |
| | | | Sarcoglycan); | |
| | | | extracellular | |
| | | | matrix proteins | |
| Limb-Girdle MD | Gene therapy/ | beta-sarcoglycan | Dystroglycan | Upper Thigh, |
| 2E | mRNA/Gene | | complex proteins | Whole Body |
| | Editing | | (e.g., alpha | |
| | | | Sarcoglycan); | |
| | | | extracellular | |
| | | | matrix proteins | |
| Limb-Girdle MD | Gene therapy/ | delta- | Dystroglycan | Upper Thigh, |
| 2F | mRNA/Gene | sarcoglycan | complex proteins | Whole Body |
| | Editing | | (e.g., alpha | |
| | | | Sarcoglycan); | |
| | | | extracellular | |
| | | | matrix proteins | |
| Limb-Girdle MD | Gene therapy/ | Fukutin related | Dystroglycan | Upper Thigh, |
| 2i, 2K, 2M, 2N, | mRNA/Gene | protein (FKRP), | complex proteins- | Whole Body |
| or 2O | Editing/protein | POMT1, | glycosylation | |
| | replacement | Fukutin, | of alpha | |
| | | POMT2, or | dystroglycan; | |
| | | POMGnT1 | extracellular | |
| | | | matrix proteins | |
| Myotonic | Muscle Blind | MNBL1 | Membrane | Upper Thigh, |
| dystrophy | (MBNL1) | | proteins, e.g., | Whole Body |
| | upregulation/release | | Chloride channel | |
| | from RNA | | (CIC-1), insulin | |
| | | | receptor | |
| Amyotrophic | Gene therapy/small | e.g., Dox7 | Neuromuscular | Upper and Lower |
| lateral sclerosis | molecule - increase | | junction proteins | limbs |
| | NMJ proteins or | | e.g., MuSK, | |
| | motor neuron activity | | LRP, neural | |
| | | | Agrin | |
| Spinal Muscular | Gene therapy/small | e.g., SMN | Neuromuscular | Upper and Lower |
| Atrophy | molecules to increase | | junction proteins | limbs |
| | SMN levels, NMJ | | e.g., MuSK, | |
| | proteins, or motor | | LRP, neural | |
| | neuron activity | | Agrin | |

The following publications provide examples of compositions and methods for therapeutic interventions in neuromuscular diseases, the entire contents of each of which is incorporated herein by reference: Systemic AAV Micro-dystrophin Gene Therapy for Duchenne Muscular Dystrophy (Mol Ther. 2018 Oct. 3; 26(10):2337-2356); Microdystrophin Gene Therapy Shows Promising Interim Results in Phase 1/2 Trial (https:musculardystrophynews.com/2018/06/22/microdystrophin-gene-therapy-shows-promise-early-trial-results/); Micro-Dystrophin Gene Therapy Goes Systemic in Duchenne Muscular Dystrophy Patients (Hum Gene Ther. 2018 July; 29(7):733-736); Nanotherapy for Duchenne muscular dystrophy (Wiley Interdiscip Rev Nanomed Nanobiotechnol. 2018 March; 10(2)); Safe and bodywide muscle transduction in young adult Duchenne muscular dystrophy dogs with adeno-associated virus (Hum Mol Genet. 2015 Oct. 15; 24(20):5880-90); Adeno-associated virus vector (AAV) microdystrophin gene therapy prolongs survival and restores muscle function in the canine model of Duchenne muscular dystrophy (DMD) (https://doi.org/10.1016/j.nmd.2015.06.458); Adeno-Associated Virus (AAV) Mediated Dystrophin Gene Transfer Studies and Exon Skipping Strategies for Duchenne Muscular Dystrophy (DMD) (Curr Gene Ther. 2015; 15(4):395-415); Dual AAV Gene Therapy for Duchenne Muscular Dystrophy with a 7-kb Mini-Dystrophin Gene in the Canine Model (Hum Gene Ther. 2018 March; 29(3):299); ClinicalTrials.gov Identifier: NCT03368742 (hppts://clinicaltrials.gov/ct2/show/NCT03368742?term=SGT⋅001&rank=1); ClinicalTrials.gov Identifier: NCT03362502 (https://clinicaltrialsgov/ct2/show/NCT03362502?term=mini-dystrophin&rank=2); ClinicalTrials.gov Identifier: NCT03375164 (https://clinicaltrials.gov/ct2/show/NCT03375164); US patent publication US2003216332, US2007073264 AA, US2009054823 AA, US2011195932 AA, US2010168072 AA, US2012009268 AA, US2012301456 AA; and patent publication GB2467560 A1.

In some embodiments, a method of evaluating efficacy of a therapeutic intervention in a subject suffering from a neuromuscular disease further comprises performing morphological imaging on the subject. In some embodiments, the morphological imaging is performed on the same body part on which the in vivo molecular imaging is performed. In some embodiments, the morphological imaging is performed on a body part or body parts that are greater than the body part that is imaged with in vivo molecular imaging. For example, a subject's upper thigh is imaged using in vivo molecular imaging to determine the level and/or localization of α-sarcoglycan, and using morphological imaging, the subject's entire leg is scanned. In some embodiments, morphological imaging is performed by the same imager that is used to perform the in vivo molecular imaging. In some embodiments, different imagers are used to perform the in vivo imaging and morphological imaging. In some embodiments, images from separate imagers are then aligned using imaging software. An example of combined in vivo molecular imaging and morphological imaging (e.g., positron emission tomography and computerized tomography) can be found in Townsend et al. (Semin Nucl Med., 2003, 33(3):193). Combined imaging, which is also herein referred to as “hybrid imaging”, can be advantageous over in vivo molecular imaging alone by providing a reference for localization of a detectable marker.

In some embodiments, in vivo molecular imaging, either with or without morphological imaging, is performed only after a therapeutic intervention is administered to a subject. In some embodiments, imaging is performed before and after a subject undergoes a therapeutic intervention (e.g., a day before the start and a day after the end of a therapeutic regimen, respectively). In some embodiments, imaging is performed on a subject a few hours to 6 months before and after a subject undergoes a therapeutic intervention (e.g., 6 months to a few hours before start and a few hours to 6 months after the end of a therapeutic intervention, 1 month to a few hours before start and a few hours to 1 month after the end of a therapeutic intervention, or 1 week to a few hours before start and a few hours to 1 week after the end of a therapeutic intervention). In some embodiments, imaging is performed 1-60 minutes before the start and/or after the end of a therapeutic regimen. It is to be understood that any combination of the intervals described above for imaging before and after a therapeutic intervention is encompassed herein. In some embodiments, imaging is performed at separate times during a regimen of a therapeutic intervention. For example, imaging to determine level and/or expression of a detectable marker can be performed before the start of a therapeutic intervention, at equally (or unequally) spaced time intervals during treatment, and/or then after treatment (e.g., after end of every month for a 12 month long therapeutic regimen, or at 6 months, 9 months and 12 months of a 12 month regimen). In some embodiments, imaging is performed after a certain time period after the end of the therapeutic intervention (for example 12 months, 18 months or 5 years after the end of a therapeutic intervention). It is to be understood that the efficacy of a therapeutic intervention can be evaluated at any time after the end of the therapeutic intervention and repeatedly. Efficacy of a therapeutic intervention can also be evaluated during the time a subject is undergoing a therapeutic regimen (e.g., 3,6 or 9 months after the start of a 1 year long therapeutic intervention).

In some embodiments, results of imaging are compared to prior results of imaging of the same subject.

In some embodiments, results of imaging on a subject after the subject undergoes a therapeutic intervention are compared to imaging results of a reference population. A reference population may be a population of subjects with the same disease, the same age group, same gender, same geographical location, same genetic background, and/or same genetic mutation/s. In some embodiments, the reference population comprises subjects with the same disease or same clinical symptoms. In some embodiments, the reference population comprises subjects with the same mutation but different disease (or symptoms). In some embodiments, a reference population comprises subjects with no disease or disease symptoms, and may be considered “normal.” In some embodiments, a reference population comprises subjects that may have other diseases but not the neuromuscular disease of the subject. In some embodiments, a reference population comprises subjects with a more severe form of the neuromuscular disease as the subject.

### Neuromuscular Disease and Dystrophies

A neuromuscular disease is a disorder that affects the peripheral nervous system or muscle. The terms “disease” and “dystrophy” are used interchangeably herein in this context.

In some embodiments, a neuromuscular disease is a muscular dystrophy, a spinal muscular atrophy, inflammatory myopathy, disease of peripheral nerve, disease of neuromuscular junction, metabolic disease of the muscle, central core disease, hyperthyroid myopathy, myotonia congenita, myotubular myopathy, nemaline myopathy, paramyotonia congenita, periodic paralysis-hypokalemic-hyperkalemic myopathy, a motor neuron disease, or frailty syndrome. In some embodiments, a neuromuscular disease is a disease or a condition that is associated with loss of muscle mass or function. In some embodiments, a neuromuscular disease is one that is associated with loss of a targetable protein, whose expression can be restored with an intervention (e.g., a therapeutic target).

In some embodiments, a muscular dystrophy is Becker muscular dystrophy, congenital muscular dystrophy, Duchenne muscular dystrophy (DMD), distal muscular dystrophy, Emery-Dreifuss muscular dystrophy, faciocarpulohumeral muscular dystrophy, limb-girdle muscular dystrophy (LGMD), myotonic muscular dystrophy or oculopharyngeal muscular dystrophy. In some embodiments, a muscular dystrophy is Becker muscular dystrophy or DMD. In some embodiments, a muscular dystrophy is DMD.

A LGMD may be a LGMD1 or LGMD2. In some embodiments, a LGMD1 is LGMD 1A, LGMD 1B, LGMD 1C, LGMD 1D, LGMD 1E, LGMD 1F, LGMD 1G or LGMD 1H. In some embodiments, a LGMD2 is LGMD2A, LGMD2B, LGMD2C, LGMD2D, LGMD2E, LGMD2F, LGMD2G, LGMD2H, LGMD2I, LGMD2J, LGMD2K, LGMD2L, LGMD2M, LGMD2N, LGMD2O or LGMD2Q.

In some embodiments, a myotonic muscular dystrophy is myotonic muscular dystrophy type 1, also called Steinert disease. In some embodiments, a myotonic muscular dystrophy is myotonic dystrophy type 2, also called proximal myotonic myopathy.

A motor neuron disease is a disease that affects motor neurons. In some embodiments, a motor neuron disease is amyotrophic lateral sclerosis (ALS), primary lateral sclerosis, progressive muscular atrophy, progressive bulbar palsy or pseudobulbar palsy. In some embodiments, a motor neuron disease is ALS.

Spinal muscular atrophies (SMAs) are disorders characterized by degeneration of lower motor neurons. In some embodiments, a SMA is a proximal spinal muscular atrophy that affects primarily proximal muscles. In some embodiments, a SMA is distal spinal muscular atrophy. In some embodiments, a SMA is an autosomal recessive proximal spinal muscular atrophy or a localized spinal muscular atrophy. In some embodiments, a SMA is an X-linked SMA (e.g., SMAX1, SMAX2 or SMAX3), a distal SMA (e.g., DSMA1, DSMA2, DSMA3, DSMA4, DSMA5, DSMAVB or distal hereditary motor neuronopathy type 7A), autosomal dominant distal SMA (e.g., distal hereditary motor neuropathy type 2A or distal hereditary motor neuronopathy type 8), congenital distal SMA, scapuloperoneal SMA, juvenile segmental SMA, Finkel-type proximal SMA, Jokela-type SMA, SMA with lower extremity predominance 1, SMA with lower extremity predominance 2, SMA with progressive myoclonic epilepsy, SMA with congenital bone fractures, SMA with pontocerebellar hypoplasia or juvenile asymmetric segmental SMA.

In some embodiments, a disease of neuromuscular junction is immune-mediated, caused by a toxin (e.g., snake venom) or congenital. In some embodiments, a disease of neuromuscular junction is presynaptic, synaptic or postsynaptic.

In some embodiments, a metabolic disease of muscle is acid maltase deficiency (Pompe disease), carnitine deficiency, carnitine palmityl transferase deficiency, debrancher enzyme deficiency (Cori or Forbes disease), lactate dehydrogenase deficiency, myoadenylate deaminase deficiency, phosphofructokinase deficiency (Tarui disease), phosphoglycerate kinase deficiency, phosphoglycerate mutase deficiency or phosphorylase deficiency (McArdle disease).

In some embodiments, frailty syndrome involves sarcopenia, cachexia or both sarcopenia and cachexia. In some embodiments, a neuromuscular disease is any condition in which sarcopenia is observed or cachexia is observed. In some embodiments, a condition associated with loss of muscle mass or function is cachexia, chronic obstructive pulmonary disease (COPD), chronic kidney disease (CKD), heart failure, aging or acute muscle atrophy cause by immobilization due to injury or surgical procedure. In some embodiments, an injury is hip fracture. In some embodiments, a surgical procedure is knee replacement or another surgical procedure on a knee, or surgery on the spine of a subject.

In some embodiments, a neuromuscular disease is DMD. In some embodiments, a neuromuscular disease is ALS.

Table 1 provides examples of neuromuscular diseases and dystrophies.

### Therapeutic Intervention

In some embodiments, a therapeutic intervention for a neuromuscular disease is disease correcting. In some embodiments, a therapeutic intervention is a pharmacologic or pharmacological regimen, which comprises administering to a subject a pharmacologic molecule (e.g., a small molecule). In some embodiments, a therapeutic intervention comprises administering a biologic or biological molecule, e.g., a protein, a peptide, an antibody or antibody fragment. In some embodiments, a biologic molecule is a composition comprising virus particles comprising a gene encoding a protein, which may be a therapeutic target. In some embodiments, a therapeutic intervention is a rehabilitative intervention (e.g., a physical exercise regimen). In some embodiments, a therapeutic intervention results in an increased expression or increased activity of a functional form of a therapeutic target. In some embodiments, a therapeutic intervention directly affects the level and/or localization of a detectable marker.

In some embodiments, a therapeutic intervention comprises more than one of the following: a pharmacologic, a biologic and/or a rehabilitative intervention. In some embodiments, a therapeutic intervention is administered to a subject for a finite time period (e.g., a day to a week, a week to a month, a month to a year, or over several years or decades). In some embodiments, a therapeutic intervention comprises administering a pharmacologic or biologic molecule 1-1000 times (e.g., 1-7, 1-14, 1-30, 1-60, 1-120, 1-280, 1-500, 1-1000 times). In some embodiments, a pharmacologic or biologic molecule is administered, or a rehabilitative regiment provided to a subject perpetually, as long as a subject lives. In some embodiments, a therapeutic intervention comprises administering a pharmacologic or biologic molecule over the course of a day to 1 year (e.g., 1 day, 3 days, 1 week, 1 month, 6 months, 9 months or 1 year). In some embodiments, a therapeutic intervention is administered to a subject for more than 1 year (e.g., 1 year, 2 years, 5 years, 10 years, 20 years or 30 years), or even perpetually as long as the subject lives.

Table 1 provides examples of different neuromuscular diseases and therapeutic interventions that can be used in some embodiments.

In some embodiments, a neuromuscular disease is DMD, and a therapeutic intervention is microdystrophin delivery (e.g., via gene therapy), exon skipping antisense oligonucleotides for a gene encoding dystrophin, stop codon read-through for a gene encoding dystrophin or gene editing for a gene encoding dystrophin. In some embodiments, a neuromuscular disease is DMD, and a therapeutic intervention increases the level of functional dystrophin in a subject, increases the level of functional utrophin in a subject, or increases the level of both functional dystrophin and functional utrophin. A therapeutic intervention that increases the level of functional utrophin in a subject may be micro-utrophin delivery using gene delivery approaches (e.g., viral vector approaches), utrophin upregulation (e.g., with compounds of the SMT C1100 family), membrane stabilization or α7 integrin modulation. Herein, α7 integrin is also referred to as integrin-α7. In some embodiments, membrane stabilization is performed by upregulation of expression of galectin-1, biglycan, α7 integrin or laminin.

For ALS, a therapeutic intervention may be a gene therapy or small molecule therapy that results in increased neuromuscular junction (NMJ) proteins or increased motor neuron activity.

For SMA, a therapeutic intervention may be a gene therapy or small molecule therapy that increases levels of survival of motor neuron (SMN), NMJ proteins (e.g., muscle-specific kinase (MuSK), leucine-responsive regulatory protein (LRP) or agrin) or increases motor neuron activity.

In some embodiments, a therapeutic gene or nucleic acid described herein can be delivered using a viral vector (e.g., using a recombinant virus such as a recombinant adeno-associated virus). However, other techniques can be used for gene or nucleic acid delivery.

Table 1 provides examples of detectable markers and therapeutic targets for some neuromuscular diseases. In some embodiments, when a neuromuscular disease is Becker muscular dystrophy or DMD, a therapeutic target is dystrophin or utrophin. In some embodiments, a neuromuscular disease is DMD, a therapeutic target is dystrophin or utrophin, and a detectable marker is α-sarcoglycan, β-sarcoglycan, γ-sarcoglycan, δ-sarcoglycan, ε-sarcoglycan, ζ-sarcoglycan, α-dystroglycan, β-dystroglycan, laminin α2, α-syntrophin, β1-syntrophin, β2-syntrophin, γ1-syntrophin, γ2-syntrophin, α-dystrobrevin, β-dystrobrevin, sarcospan, calveolin-3 or neuronal nitric oxide synthase (nNOS). In some embodiments, a neuromuscular disease is DMD and a therapeutic target is dystrophin. In some embodiments, a neuromuscular disease is DMD and a therapeutic target is utrophin. In some embodiments, a neuromuscular dystrophy is DMD or Becker muscular dystrophy and a detectable marker is any protein of the DGC or a protein that associates with a member of the DGC.

In some embodiments, a neuromuscular disease is DMD, Becker muscular dystrophy or a LGMD, and a detectable marker is a member of dystrophin-associated glycoprotein complex (DGC), or an extracellular matrix protein that binds to any member of DGC. In some embodiments, a neuromuscular disease is DMD, Becker muscular dystrophy or a LGMD, and a detectable marker is any of the following proteins: α-sarcoglycan, β-sarcoglycan, γ-sarcoglycan, δ-sarcoglycan, ε-sarcoglycan, ζ-sarcoglycan, α-dystroglycan, β-dystroglycan, laminin α2, α-syntrophin, βsyntrophin, β2-syntrophin, γ1-syntrophin, γ2-syntrophin, α-dystrobrevin, β-dystrobrevin, sarcospan, calveolin-3, and neuronal nitric oxide synthase (nNOS).

In some embodiments, a neuromuscular disease is DMD or Becker muscular dystrophy, a therapeutic target is dystrophin or utrophin, and a detectable marker is α-sarcoglycan, γ-sarcoglycan, α-dystroglycan, β-dystroglycan or laminin α2.

In some embodiments, a neuromuscular disease is LGMD1A and a therapeutic target is myotilin. In some embodiments, a neuromuscular disease is LGMD1B and a therapeutic target is lamin A/C. In some embodiments, a neuromuscular disease is LGMD1C and a therapeutic target is caveolin-3. In some embodiments, a neuromuscular disease is LGMD2A and a therapeutic target is calpain 3. In some embodiments, a neuromuscular disease is LGMD2B and a therapeutic target is dysferlin. In some embodiments, a neuromuscular disease is LGMD2C and a therapeutic target is γ-sarcoglycan. In some embodiments, a neuromuscular disease is LGMD2D and a therapeutic target is α-sarcoglycan. In some embodiments, a neuromuscular disease is LGMD2E and a therapeutic target is β-sarcoglycan. In some embodiments, a neuromuscular disease is LGMD2F and a therapeutic target is δ-sarcoglycan. In some embodiments, a neuromuscular disease is LGMD2G and a therapeutic target is telethonin. In some embodiments, a neuromuscular disease is LGMD2I, LGMD2K, LGMD2M, LGMD2N or LGMD2O and a therapeutic target is a Fukutin kinase related protein (FKRP), POMT1, Fukutin, POMT2 or POMGnT1. For any of the neuromuscular diseases LGMD2I, LGMD2K, LGMD2M, LGMD2N or LGMD2O, a therapeutic intervention may be delivery of the following proteins: Fukutin related protein (FKRP), POMT1, Fukutin, POMT2, POMGnT1, or Glycosyltransferase-like protein LARGE1 (LARGE). In some embodiments, a therapeutic intervention may be delivery of genes encoding any of the following proteins: Fukutin related protein (FKRP), POMT1, Fukutin, POMT2, POMGnT1, or Glycosyltransferase-like protein LARGE1 (LARGE). In some embodiments, a neuromuscular disease is LGMD2I, LGMD2K, LGMD2M, LGMD2N or LGMD2O and a detectable marker is glycosylated α-dystroglycan. In some embodiments, a neuromuscular disease is LGMD2I and a therapeutic target is FKRP. In some embodiments, a neuromuscular disease is LGMD2K and a therapeutic target is POMT1. In some embodiments, a neuromuscular disease is LGMD2J and a therapeutic target in titin or calpain-3, or both titin and clapain-3. In some embodiments, a neuromuscular disease is LGMD2L and a therapeutic target in fukutin. In some embodiments, a neuromuscular disease is LGMD2N and a therapeutic target in POMT2. Therapeutic targets of different LGMD subtypes are discussed by Norwood et al. (European J of Neurology, 2007, 14:1305) and are herein incorporated by reference.

In some embodiments, a neuromuscular disease is myotonic muscular dystrophy and a therapeutic target is muscleblind-like 1(MBNL1). For myotonic muscular dystrophy, a detectable marker is a membrane protein (e.g., chloride channel (CIC-1) or insulin receptor) in some embodiments. In some embodiments, myotonic muscular dystrophy is treated by interventions that result in upregulation of MBNL1 expression or MBNL1 release from RNA.

For ALS, a therapeutic target may be Dox7. A detectable marker for ALS may be a neuromuscular junction protein (e.g., muscle-specific kinase (MuSK), leucine-responsive regulatory protein (LRP) or agrin).

For SMA, a therapeutic target can be SMN, and a detectable marker can be a NMJ protein (e.g., muscle-specific kinase (MuSK), leucine-responsive regulatory protein (LRP) or agrin).

It is to be understood that multiple therapeutic targets can be targeted by one or more therapeutic interventions. For example two therapeutic interventions targeting dystrophin and utrophin may simultaneously be administered to a subject with DMD. In some embodiments, two therapeutic interventions target the same therapeutic target. In some embodiments, multiple therapeutic interventions are staggered with some or no overlap.

In some embodiments, any of the methods disclosed herein (either of evaluating efficacy of a therapeutic intervention or of monitoring a neuromuscular disease) further comprises either continuing the existing therapeutic intervention, changing or amending the existing therapeutic intervention, or stopping the existing therapeutic intervention. In some embodiments, any of the methods disclosed herein further comprises directing or recommending either continuing the existing therapeutic intervention, changing or amending the existing therapeutic intervention, or stopping the existing therapeutic intervention. For example, if the existing therapeutic intervention that is being administered to a subject is found to be effective in treating or managing the neuromuscular disease, that existing intervention may be continued. In some embodiments, the existing therapeutic intervention is changed or amended so that another therapy is added to the therapeutic intervention (e.g., if the existing therapeutic intervention consist of administering of a first pharmacological agent or molecule, a change in the existing therapeutic intervention may include either addition of a second pharmacological agent or molecule, or replacing the first pharmacological agent with the second pharmacological agent). In some embodiments, an existing therapeutic intervention consisting of administering of one or more pharmacological agents is supplemented with physical therapy depending on the evaluation of the efficacy of existing therapeutic intervention or monitoring of the neuromuscular disease in a subject. In some embodiments, a subject being evaluated by any one of the methods disclosed herein is not receiving any therapeutic intervention, and any one of the methods disclosed herein further comprises administering or recommending a therapeutic intervention.

### Imaging

Techniques to perform in vivo molecular imaging as well as techniques for morphological imaging are known in the art.

“Molecular imaging” as used herein refers to imaging of a subject or part of a subject (e.g., a limb) that provides information of where in the subject a particular molecule or group of molecules (e.g., 2, 3, 4, 5, 6, 7, 8, 9, 10, or more molecules) is located and/or the amount of molecule or group of molecules. In some embodiments, molecular imaging provides the location of a molecule or group of molecules in the subject's body. In some embodiments, molecular imaging provides the amount or level of a molecule or group of molecules. In some embodiments, molecular imaging provides the amount or level of a molecule or group of molecules in a particular location of the subject body (e.g., how much dystrophin is expressed in the extracellular matrix). In some embodiments, molecular imaging provides the location and/or amount of a molecule or group of molecules relative to a morphological image of the subject or part of the subject.

In vivo molecular imaging can be performed by a number of imaging techniques. Non-limiting examples of molecular imaging techniques include positron emission tomography (PET), single-photon emission computer tomography (SPECT), magnetic resonance imaging (MRI), targeted contrast enhanced ultrasound (targeted CEUS) imaging or optical coherence tomography (OCT). In vivo molecular imaging is used to detect a detectable marker by using an imaging agent. An imaging agent is an antibody, a protein, a peptide, a small molecule, an antibody-derived construct (e.g., scfv, Fab or Fab2′), an adnectin, an aptamer, a nanobody or a protein domain that has suitable affinity and specificity to a detectable marker to be detected. An imaging agent is conjugated to a tracer. A tracer is a molecule that provides or is capable of providing a detectable signal when used in conjunction with a particular imaging modality (e.g., PET or SPECT). Examples of detectable signals are fluorescent signals, radiochemical signals (e.g., gamma rays) or a radio frequency energy. In some embodiments, a tracer modifies a detectable signal. For example, gas filled microbubbles modulate how sound waves are reflected in CEUS. Therefore, a tracer can be a radioisotope, a fluorophore, a paramagnetic agent or an echocontrast agent. Depending on the imaging modality used, an imaging agent is conjugated to a PET tracer, a SPECT tracer, a paramagnetic contrast agent, an ultrasound contrast agent or an OCT contrast agent.

Non-limiting examples of a PET tracer are carbon-11, nitrogen-13, oxygen-15, fluorine-18, indium-11, gallium-68, copper-64, zirconium-89, iodine-125 and rubidium-82. In some embodiments, instrumentation that allows for time-of-flight PET is used. Immuno-PET is discussed by Wu (J Nucl Med., 2009, 50:2), and is incorporated in its entirety herein.

In some embodiments, a SPECT tracer is iodine-123, technetium-99m, xenon-133, thallium-201 or fluorine-18.

Non-limiting examples of paramagnetic contrast agents for use with MRI are gadoterate, gadodiamide, gadobenate, gadopentetate, gadoteridol, gadofosveset, gadoversetamide, gadoxetate, gadobutrol and ferumoxsil.

Contrast agents for ultrasound imaging, also referred to as echocontrast agents, may comprise microbubbles. In some embodiments, microbubbles comprise perfluorocarbon or nitrogen gas. In some embodiments, an ultrasound contrast agent is SonoVue, Optison, Levovist, Perflexane, lipid microspheres or perflutren lipid microspheres. In some embodiments, targeted contrast-enhanced ultrasound (targeted CEUS) is used to detect a detectable marker.

In some embodiments, gold nanorods (e.g., polyethylene-glycol-coated gold nanorods) are used as contrast agents for OCT. Variations of OCT may include photothermal OCT (Tucker-Schwartz and Skala, Biomedical Optics & Medical Imaging, 2013, SPIE Newsroom) and doppler OCT (Wang et al, PLoS One, 2014 9(3):e90690). Variations in gold nanoparticles for OCT are discussed by Al Rawashdeh et al. (Journal of Nanoparticle Research, 2012, 14:1255) and are incorporated by reference herein in their entirety.

The choice of imaging modality for in vivo molecular imaging depends on multiple factors including, but not limited to sensitivity, temporal and spatial resolution, quantifiability, acquisition time, simplicity, availability and relative risk for the subject being imaged. Different imaging modalities with respect to these factors are discussed in Chen et al. (Biomed Res Int. 2014; 2014: 819324), Pysz et al. (Clin Radiol. 2010 July; 65(7): 500-516) and Barsanti et al. (World J Diabetes. 2015 Jun. 25; 6(6): 792-806), which are incorporated herein by reference in their entirety.

In some embodiments, an imaging agent will have an affinity to a detectable marker as defined by a dissociation constant K_{d} of 1×10⁻⁴M-1×10⁻¹⁸M (e.g., 1×10⁻⁵M-1×10⁻¹⁵M, 1×10⁻⁶M-1×10⁻¹⁵M, 1×10⁻⁷M-1×10⁻¹⁴M, 1×10⁻⁸M-1×10⁻¹³M, 1×10⁻⁹M-1×10⁻¹²M, 1×10⁻¹⁰M-1×10⁻¹¹M, 1×10⁻⁶M-1×10⁻¹²M or 1×10⁻⁶M-1×10⁻⁹M). Binding constants can be measured in a number of different ways and experimental methods to measure binding and calculate binding constants using different platforms (e.g., Bioacore, Octet or using radiochemical or other labeling technique) are known in the art. In some embodiments, the specificity of an imaging agent for binding to a detectable marker is defined such that it binds to a detectable marker with a binding dissociation constant that is 2−1×10¹⁸ times lower than the binding constant for binding of the imaging agent to other proteins or tissue components. For example an imaging agent may bind to a detectable marker with a binding constant that is at least 2-10 times (e.g., 2-3, 2-5, 4-6, 5-10 or 8-10 times), at least 10-100 times (e.g., 10-20, 20-50, 50-80 or 80-100 times), at least 100-1000 times (e.g., 100-200, 200-500, 500-800 or 800-1000 times) or at least 1×10³-1×10¹⁸ times (e.g.,1×10³-1×10⁹, 1×10⁹-1×10¹², 1×10¹²-1×10¹⁵, or 1×10¹⁵-1×10¹⁸ times) lower than its binding constant to other proteins or tissue components. In some embodiments, an imaging agent is a nanoparticle. Nanoparticles for use in imaging are described by Padmanabhan et al. (Acta Biomater. 2016, S1742-7061(16)30271-9) and are herein incorporated by reference in their entity.

In some embodiments, an imaging agent is administered to a subject intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs by direct injection. In some embodiments, an imaging agent is administered to a subject intravenously. In some embodiments, an imaging agent is administered to a subject by direct injection into a tissue to be imaged.

“Morphological imaging” as used herein refers to imaging of a subject or part of a subject (e.g., a limb) that provides morphological information of a subject, e.g., the size and location of a particular tissue or organ, or a group of cells, or a single cell. Morphological imaging can be performed by MRI, ultrasound or computerized tomography (CT). In some embodiments, a contrast agent is used to improve the visibility of internal body structures. Paramagnetic contrast agents for use with MRI are described above. In some embodiments, untargeted contrast-enhanced ultrasound is used for morphological imaging. Ultrasound contrast agents that can be used in untargeted contrast-enhanced ultrasound are described above.

Imaging is performed on either an organ or region of a subject that is particularly affected by a neuromuscular disease, or whole body imaging can be performed. In some embodiments, a subject's upper thigh is imaged. In some embodiments, one or all of a subject's upper or lower limbs are imaged. Table 1 provides examples of regions of the body that are particularly affected by certain neuromuscular diseases. In some embodiments, when both molecular and morphological imaging is performed, the same region of a subject's body is imaged by in vivo molecular imaging and morphological imaging. In some embodiments, there is a partial overlap of the region being imaged by molecular and morphological imaging.

In some embodiments, morphological imaging is used to provide a background for information obtained via molecular imaging. Accordingly, in some embodiments of any one of the methods described herein, images obtained via molecular imaging are overlayed or superimposed on images obtained via morphological imaging. Such overlaying or superimpostions can be performed either manually or by using imaging software. In some embodiments, the superimposition of molecular images and morphological images provides location of molecules of interest within morphological structures.

In some embodiments, a subject is human. In some embodiments, a subject is a mouse (e.g., mdx mouse model for DMD), a rat, a dog (e.g., GRMD dog model for DMD), a cat, a non-human primate, a horse, a cow, a donkey or a rabbit. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, a subject is an adult (e.g., a human adult). In some embodiments, a subject is a child (e.g., a human child).

### Detection of Multiple Detectable Markers

In some embodiments, a plurality (e.g., 2, 3, 4 or 5) of detectable markers are detected using in vivo molecular imaging. In some embodiments, a plurality of detectable markers colocalize in tissues. In some embodiments, a plurality of detectable markers are involved in disease etiology but do not colocalize. In some embodiments, the same imaging modality is used to detect a plurality of detectable markers. In some embodiments, more than one imaging modality is used to detect multiple detectable markers. For example, PET can be used to detect a first detectable marker and SPECT to detect a second detectable marker. In some embodiments, when multiple detectable markers are detected using the same imaging modality, the same imaging agents are used (e.g., carbon-11 as a PET tracer to detect α- and (β-sarcoglycan in DMD). In some embodiments, when multiple detectable markers are detected using the same imaging modality, different imaging agents are used (e.g., carbon-11 as a PET tracer to detect α-sarcoglycan and nitrogen-13 to detect (β-sarcoglycan). In some embodiments, imaging to detect a first detectable marker is immediately followed by imaging to detect a second detectable marker. In some embodiments, detection of the second detectable marker is performed after an imaging agent conjugated to a tracer that targets the first detectable marker has passed from the body.

A Method of Monitoring Neuromuscular Disease

In some aspects, provided herein is a method of monitoring a disease, the method comprising determining level and/or localization of a detectable marker by performing in vivo molecular imaging on a subject. In some embodiments, level and/or localization of the detectable marker is modulated by a therapeutic target if the subject is undergoing a therapeutic intervention. In some embodiments, the detectable marker is different from the therapeutic target. In some embodiments, a detectable marker and a therapeutic target is the same.

In some embodiments, a therapeutic target is a protein that influences (e.g., causes) neuromuscular disease or symptoms of a neuromuscular disease. In some embodiments, a method of monitoring neuromuscular disease also comprises performing morphological imaging on a subject with neuromuscular disease. In some embodiments, imaging is performed to determine level and/or localization of a detectable marker in a subject at two or more time points (separated by hours, days, months or years). For example, imaging may be repeated one week to 80 years (e.g., one week tol month, 1 month to 1 year, 1 year to 5 years, 5 years to 10 years, 10 years to 30 years, 10 years to 50 years, 10 years to 80 years) after imaging was performed the first time. In some embodiments, imaging to determine level and/or localization of a detectable marker is performed at regular intervals after diagnosis (e.g., once every 3 months, once a year, once every two years, or once every 5-10 years).

In some embodiments, level and/or localization of a detectable marker is determined in more than one subject (e.g., 2-10, 10-20, 20-50, 50-100 or more than 100 subjects) with different disease severities. A database of such measurements can be used to assess the severity of disease for a particular subject either within or outside the population of subjects for which the level and/or localization of a detectable marker is determined. In some embodiments, level and/or localization of a detectable marker is determined in subjects with different genetic abnormalities. A database of such measurements can be used to assess disease prognosis in a patient with a particular genetic abnormality.

In some embodiments, level and/or localization of a detectable marker is determined after a therapeutic candidate has been administered to a subject in order to determine the efficacy of the therapeutic candidate on clinical improvement. In some embodiments, level and/or localization of a detectable marker is determined both before and after administering a therapeutic candidate to a subject. In some embodiments, level and/or localization of a detectable marker in a subject is compared to level and/or localization of the detectable marker in a reference population of subjects. Reference populations are discussed above.

In some embodiments, to assess disease progression, imaging is performed on multiple subjects with neuromuscular disease to determine level and/or localization of a detectable marker at multiple time points.

It is to be understood that the methods disclosed herein can also be used to discover and develop biomarkers for neuromuscular diseases that may and may not be used in conjunction with a therapeutic intervention.

It should be understood that any one of the methods of monitoring a neuromuscular disease as described herein is, in some embodiments, used to determine whether a subject should be provided a therapeutic intervention or determine the type of therapeutic intervention to be provided to the subject. A therapeutic intervention may be any one of the therapeutic interventions for a neuromuscular disease described above. Accordingly in some embodiments, provided herein is a method of monitoring a neuromuscular disease in a subject using any one of the methods described herein, and then providing a therapeutic intervention to the subject based on the result of the method of monitoring the neuromuscular disease. In some embodiments, a therapeutic intervention is a pharmacologic or pharmacological regimen, which comprises administering to a subject a pharmacologic molecule (e.g., a small molecule). In some embodiments, a therapeutic intervention comprises administering a biologic or biological molecule, e.g., a protein, a peptide, an antibody or antibody fragment.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Evaluation of a Therapeutic Intervention for Duchenne Muscular Dystrophy (DMD) using Non-Invasive Detection

Duchenne muscular dystrophy (DMD) is a genetic disorder characterized by progressive muscle degradation and weakness. Disease symptoms start in early childhood and are first manifested in muscles of the hips, pelvic area, thighs and shoulders. Late symptoms manifest in skeletal (voluntary) muscles in the arms, legs and trunk. Children with DMD generally show weakness of heart and respiratory muscles in their early teens and rarely survive to live the third decade of life.

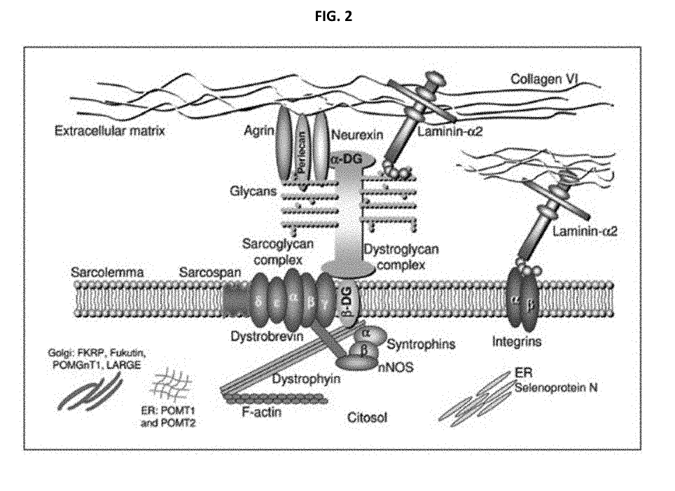

DMD is caused by an absence of dystrophin in muscles. Dystrophin is a protein with both a structural and signaling role in muscle. It is localized to the cytoskeleton immediately beneath sarcolemma. In patients with DMD, the open reading frame of the X-linked DMD gene, encoding dystrophin, is disrupted by deletions, duplications, point mutations and other smaller rearrangements (Lancet Neurol, 2010, 9: 77). Dystrophin connects the subsarcolemmal F-actin cytoskeleton to β-dystroglycan (β-DG), which is associated with α-dystroglycan (α-DG) and in turn connects to proteins of the extracellular matrix (FIG. 2). By associating with other proteins, dystrophin forms the dystrophin-associated glycoprotein complex (DGC), which consists of three subcomplexes. The first subcomplex comprises the sarcoglycans (e.g., α-, (β-, γ, and δ-sarcoglycan). The second subcomplex comprises syntrophin, neuronal nitric oxide synthase (nNOS) and dystrobrevin. The third subcomplex comprises of BDG and α-dystroglycan. Absence of dystrophin and destabilization of the DGC results in stretch-induced damage of muscles and increased intracellular calcium influx, which can lead to necrosis of skeletal and cardiac muscle fiber, inflammation and replacement of muscle with fibro-adipose tissue. Dystrophin is also involved in the localization of nNOS, which regulates blood flow in skeletal muscle.

### Therapeutic Interventions for DMD

While current therapies are symptomatic, several experimental therapeutic interventions are in development. Exon skipping therapy induces skipping of dystrophin exon 51 in patients using splice switching oligomers that transform out-of-frame mutations at the mRNA level. For some DMD patients who have a nonsense mutation resulting in a premature stop codon in the corresponding mRNA of dystrophin, termination codon read through strategies (e.g., using small molecules such as ataluren) is also a possible therapeutic intervention. Dystrophin gene replacement or editing therapies, and non-dystrophin strategies also exist. Delivery of microdystrophin gene using adeno associated virus is a promising therapeutic intervention for DMD (Shin et al., Mol Ther., 2013 4:750).

Therapeutic interventions that are aimed at utrophin expression can be applicable to all DMD patients, regardless of the dystrophin mutation. Utrophin modulation strategies include direct delivery of the protein, stabilization of the protein by restoration of other protein complexes, or stabilization of RNA, gene delivery approaches (e.g., viral approaches), and compounds that modulate utrophin expression at the transcriptional level (e.g., compounds of the SMT C1100 family).

### Subjects and Experimental Procedure

A subject in this experiment is a human patient with DMD, an mdx mouse that has a nonsense point mutation in exon 23 that aborts full-length dystrophin expression, a utrophin/dystrophin double-knockout mouse, an α7 integrin/dystrophin double-knockout mouse, any of the known dystrophin-deficient dogs (e.g., GRMD model), or any other known animal model for DMD (McGreevy et al., Disease Models and Mechanisms,2015, 8: 195).

Methods of administering a therapeutic intervention to a subject will vary depending on the therapeutic intervention and the subject species, but are known in the art. For example, a dystrophin splice-switching oligomer, such as eteplirsen, is administered to human subjects by injecting eteplirsen into one extensor digitorum brevis muscle whereas the contralateral muscle receives a saline injection (Cirak et al., Mol Ther., 2012, 2: 462). Utrophin modulators are administered to mice by intraperitoneal injection, oral gavage, or a combination of both methods (Guiraud et al., Human Molecular Genetics, 2015, 24: 15). A mouse can be administered microdystrophin by injection of adeno-associated virus particles via the tail vein. A dog can be administered microdystrophin by injection of adeno-associated virus particles into ECU muscles (Shin et al., Mol Ther., 2013 4:750).

Imaging is performed in varying configurations and involves either a non-invasive imaging modality to detect the detectable marker alone, or an imaging modality to detect the detectable marker in combination with some form of morphological imaging.

For therapeutic interventions targeting dystrophin or utrophin, the following proteins are targeted for detection: α-sarcoglycan, β-sarcoglycan, γ-sarcoglycan, δ-sarcoglycan, ε-sarcoglycan, ζ-sarcoglycan, α-dystroglycan, β-dystroglycan, laminin α2, α-syntrophin, β1-syntrophin, β2-syntrophin, γ1-syntrophin, γ2-syntrophin, α-dystrobrevin, β-dystrobrevin, sarcospan, calveolin-3, and neuronal nitric oxide synthase (nNOS). The level and localization of these proteins is detected using an imaging agent that is an antibody, a protein, a peptide, a small molecule, an antibody-derived construct, an adnectin, an aptamer, a nanobody or a protein domain that binds with affinity and specificity to the detectable marker. In some configurations, dystrophin is the therapeutic target as well as the detectable marker. Similarly, utrophin, in some configurations, is the therapeutic target as well as the detectable marker. The imaging agent is conjugated to a tracer, the type of which depends on the imaging modality.

In some configurations, an in vivo molecular imaging modality to measure the level and/or localization of the detection target is used alone, and in other configurations, it is combined with morphological imaging. This combination of two imaging modalities enables determination of localization of the tracer and quantification of restoration of the DGC stability following treatment. Non-invasive in vivo molecular imaging is done using PET, SPECT, MRI, ultrasound or OCT. Morphological imaging is done using MRI or CT. Accordingly, imaging agents are conjugated to a PET tracer (e.g., carbon-11), a SPECT tracer (e.g., xenon-133), a paramagnetic contrast agent (e.g., gadoxetate), an ultrasound contrast agent (e.g., SonoVue, Optison, or Levovist microbubbles), or an OCT contrast agent (e.g., gold nanoparticles). Method of performing combined imaging using an in vivo molecular imaging modality with a morphological imaging modality are known in the art. For example, Townsend et al. (Semin Nucl Med., 2003, 33(3):193) describe combined PET and CT imaging.

Imaging is performed on a subject before and after administering a regimen of therapeutic intervention. For example, in one configuration, a human subject with DMD is imaged using CT and PET to determine the level and/or localization of α-sarcoglycan in an upper thigh. In another configuration, whole body imaging is performed. Thereafter, the subject is administered a single intravenous injection of adeno-associated virus (AAV) to systemically deliver nucleic acid that encodes shortened, but functional form of the dystrophin protein, described as a micro- or mini-dystrophin. At a later date (e.g., 4-12 weeks), when dystrophin expression is anticipated, the subject's upper thigh is then imaged again. Alternatively, AAV comprising nucleic acid encoding micro- or mini-dystrophin is administered locally via intramuscular injection.

### Results and Summary

As shown in FIG. 1A, before treatment of the DMD subject with microdystrophin, because dystrophin (illustrated by cylinder) was absent and the DGC destabilized, α-sarcoglycan could not be detected. After treating the subject with microdystrophin, α-sarcoglycan can be detected in the muscle of the upper thigh, and more particularly at the membranes of muscle cells. A comparison of the images taken of the subjects upper thigh before and after treatment indicates that dystrophin is at least partially restored to the DGC. Comparing the imaging data to clinical symptomatic data for the subject indicates that detection of α-sarcoglycan can serve as a marker for better muscle quality in the subject.

This example discusses non-invasive in vivo methodologies and imaging strategy configurations that allow simple and effective evaluation of the efficacy of a therapeutic intervention to improve muscle quality. However, it is to be understood that the techniques discussed in this example can be adapted to assess the efficacy of any therapeutic intervention for DMD, and can be applied to other muscular dystrophies and neuromuscular diseases.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method of evaluating efficacy of a therapeutic intervention in a subject suffering from a neuromuscular disease, the method comprising:
determining level and/or localization of a detectable marker comprising performing in vivo molecular imaging on the subject after the subject undergoes the therapeutic intervention;
wherein the level and/or localization of the detectable marker is modulated by a therapeutic target of the therapeutic intervention.

2. The method of claim 1, further comprising performing morphological imaging on the subject.

3. (canceled)

4. The methods of claim 1, further comprising performing in vivo molecular imaging on the subject before the subject undergoes the therapeutic intervention.

5-8. (canceled)

9. The method of claim 1, wherein the therapeutic target is a cytosolic protein, a membrane-bound protein, a glycoprotein or a matrix-anchoring protein.

10. (canceled)

11. The method of claim 1, wherein the neuromuscular disease is one of the following: muscular dystrophy, spinal muscular atrophy, inflammatory myopathy, disease of peripheral nerve, disease of neuromuscular junction, metabolic disease of muscle, central core disease, hyperthyroid myopathy, myotonia congenita, myotubular myopathy, nemaline myopathy, paramyotonia congenita, periodic paralysis-hypokalemic-hyperkalemic myopathy, motor neuron disease, frailty syndrome or a condition associated with loss of muscle mass or function.

12. The method of claim 11, wherein the muscular dystrophy is Becker muscular dystrophy, congenital muscular dystrophy, Duchenne muscular dystrophy (DMD), distal muscular dystrophy, Emery-Dreifuss muscular dystrophy, faciocarpulohumeral muscular dystrophy, limb-girdle muscular dystrophy (LGMD), myotonic muscular dystrophy, or oculopharyngeal muscular dystrophy.

13-15. (canceled)

16. The method of claim 12, wherein the muscular dystrophy is Duchenne muscular dystrophy (DMD).

17-20. (canceled)

21. The method of claim 1, wherein the therapeutic intervention is a pharmacologic, biologic, or rehabilitative therapeutic intervention that results in the increased expression or increased activity of a functional form of the therapeutic target.

22. The method of claim 21, wherein the pharmacologic therapeutic intervention comprises administering to the subject a small molecule, and/or the biologic therapeutic intervention comprises administering to the subject a gene or protein.

23-24. (canceled)

25. The method of claim 16, wherein the therapeutic target is dystrophin.

26. The method of claim 25, wherein the therapeutic intervention is microdystrophin delivery, exon skipping antisense oligonucleotides for a gene encoding dystrophin, termination codon read through strategy dystrophin, or gene editing for a gene encoding dystrophin.

27-46. (canceled)

47. The method of claim 1, wherein the in vivo molecular imaging is selected from the group consisting of: positron emission tomography (PET), single-photon emission computer tomography (SPECT), magnetic resonance imaging (MRI), targeted contrast enhanced ultrasound (targeted CEUS) imaging and optical coherence tomography (OCT).

48. The method of claim 2, wherein the morphological imaging is magnetic resonance imaging (MRI) or computerized tomography (CT).

49. The method of claim 1, wherein the in vivo molecular imaging and morphological imaging is performed on the subject's upper or lower limb, upper thigh, or whole body.

50-54. (canceled)

55. A method of monitoring a neuromuscular disease, comprising:
determining level and/or localization of a detectable marker comprising performing in vivo molecular imaging on a subject;
wherein the level and/or localization of the detectable marker is modulated by a therapeutic target of a therapeutic intervention, and;

56. The method of claim 55, further comprising performing morphological imaging on the subject.

57. The method of claim 55, further comprising determining the level and/or localization of the detectable marker in the subject at two or more time points.

58. The method of claim 55, further comprising determining the level and/or localization of the detectable marker in additional subjects with the same or different genetic abnormalities.

59. The method of claim 55, further comprising determining the level and/or localization of the detectable marker after a therapeutic candidate has been administered to the subject, to determine the efficacy of the therapeutic candidate on clinical improvement.

60. The method of claim 2, wherein the in vivo molecular imaging and morphological imaging is performed on the subject's upper or lower limb, upper thigh, or whole body.
